(19) United States
(12) Patent Application Publication
(10) Pub. No.: US 2003/0212262 A1
(43) Pub. Date: Nov. 13, 2003

(54) Human serine racemase

(76) Inventors: Thomas M Connolly, Lansdale, PA (US); Yuan Liu, Lansdale, PA (US); Menghang Xia, Blue Bell, PA (US)

Correspondence Address: MERCK AND CO INC, P O BOX 2000, RAHWAY, NJ, 070650907

(21) Appl. No.: 10/240,800

(22) PCT Filed: Apr. 2, 2001
(86) PCT No.: PCT/US01/10662

(51) Int. Cl. C12Q 1/68; A01K 67/00; C07H 21/04; C12N 9/90; C12P 13/04

(52) U.S. Cl. 536/23.2; 435/233; 435/69.1; 435/320.1; 435/325; 435/106; 800/8; 435/6

(57) ABSTRACT

The present invention provides polynucleotides and polypeptides of a human serine racemase. The polynucleotides and polypeptides are used to further provide expression vectors, host cells comprising the vectors, probes and primers, antibodies against the serine racemase protein and polypeptides thereof, assays for the presence or expression of serine racemase and assays for the identification of compounds that interact with serine racemase and transgenic animals expressing human serine racemase.

### CROSS-REFERENCE TO RELATED APPLICATIONS

This application claims the benefit of U.S. Provisional Application No. 60194,451, filed Apr. 4, 2000, the contents of which are incorporated herein by reference in their entirety.

### STATEMENT REGARDING FEDERALLY-SPONSORED R&D

Not applicable.

### REFERENCE TO MICROFICHE APPENDIX

Not applicable.

### FIELD OF THE INVENTION

The invention relates to human serine racemase, polynucleotides encoding the enzyme and assays that measure the production of racemization of serine by human serine racemase.

### BACKGROUND OF THE INVENTION

Preventing activation of the N-methyl-D-aspartate (NMDA) receptor is considered a potential therapeutic method for several clinical indications including: stroke, epilepsy, chronic pain, Parkinson's and Huntington's diseases, depression, anxiety, and glaucoma. There are two agonist binding sites on NMDA receptors-glutamate and glycine sites-and both must bind agonists to activate the receptor. Strategies to block activation include the use of competitive glutamate site antagonists and the use of receptor ion channel blockers. An alternative approach, to antagonize activation of the receptor by blocking the glycine site, is also promising and has been associated with reduced side effects when compared with glutamate site antagonists.

Serine racemase is the enzyme which catalyzes the conversion of L-serine to D-serine. In vivo, D-serine is understood to function as a co-agonist for the activation of the NMDA receptor complex by selectively binding to the glycine ligand site (Ivanovic, et al., 1998; Miyazaki, et al., 1999). In contrast to glycine, D-serine only activates the strychnine-insensitive site, but not the strychnine-sensitive site.

High concentrations of D-serine have been detected in the mammalian central nerve systems, including the human neurosystem (Hashimoto and Oka, 1997). Immunohistochemical and in situ hybridization studies reported that in brain the distribution of D-serine correlates with the expression of NMDA receptors (NR2A/NR2B) better than that of glycine (Schell et al., 1997a, 1997b).

NMDA receptors, such as NR2A and NR2B, are highly permeable to calcium. Under pathological conditions, such as stroke, and in some neuronal diseases, a large release of glutamate causes the release of D-serine from astrocytes and prolonged activation of NMDA receptors. This cascade can often lead to neuronal cell death due to the overload of calcium inside the cells.

Fluctuations of D-serine concentrations play an important role in determining the magnitude of NMDA receptor activation during physiological and pathological processes (Dalkara et al., 1990). The selective removal of endogenous D-serine by application of D-amino acid oxidase was reported to greatly reduced NMDA receptor activation in brain slice studies and in cell culture preparations (Wolosker et al., 1999). This finding indicates that reduction of D-serine levels can suppress the activation of NMDA receptors.

Because serine racemase is a key regulator of D-serine concentration in cells, the inhibition of this enzyme is expected to reduce the concentration of D-serine available to activate NMDA receptors. Regulation of the receptor ligand, rather than antagonism at a site on the receptor itself, has the potential advantage of being an upstream regulation point and thus may be easier to control.

Recently a murine serine racemase has been cloned and expressed (Wolosker et al., 1999). The murine serine racemase is a protein of 339 amino acids with a predicted molecular weight of 36.3 kDa. Western blot analysis revealed a single band protein at about 38 kDa. There is a pyridoxal-5′ phosphate (PLP) binding region in serine racemase, which is a member of PLP-dependent amino acid racemases. PLP is required for its activity (Wolosker et al., 1999b). However, the regulation of this enzyme during physiological and pathological conditions is not presently understood.

### SUMMARY OF THE INVENTION

The present invention provides polynucleotides encoding a human serine racemase, recombinant host cells containing serine racemase polynucleotides, serine racemase polypeptides, and methods of using the polynucleotides, polypeptides and host cells to conduct assays of serine racemase activity.

In particular, recombinant polynucleotides and recombinant polypeptides of human serine racemase, are provided. The recombinant serine racemase enzyme is catalytically active in the racemization of serine. The enzyme is used in in vitro and whole cell assays to screen for compounds that alter the activity of the serine racemase or interact with enzyme, or alter the expression of serine racemase. The invention includes the recombinant polynucleotides, recombinant proteins encoded by the polynucleotides, host cells expressing the recombinant enzyme and extracts prepared from host cells expressing the recombinant enzyme, probes and primers, and the use of these molecules in assays.

An aspect of this invention is a polynucleotide having a sequence encoding a serine racemase protein, or a complementary sequence. In a particular embodiment the encoded protein has a sequence corresponding to SEQ ID NO:2. In other embodiments, the encoded protein can be a naturally occurring mutant or polymorphic form of the protein. In preferred embodiments the polynucleotide can be DNA, RNA or a mixture of both, and can be single or double stranded. In particular embodiments, the polynucleotide is comprised of natural, non-natural or modified nucleotides. In some embodiments, the internucleotide linkages are linkages that occur in nature. In other embodiments, the internucleotide linkages can be non-natural linkages or a mixture of natural and non-natural linkages. In a most preferred embodiment, the polynucleotide has the coding sequence contained in sequence SEQ ID NO:1. In another preferred embodiment the polynucleotide has an equivalent sequence of a naturally occurring mutant or polymorphic serine racemase polypeptide.

An aspect of this invention is a polynucleotide having a sequence of at least about 25 contiguous nucleotides that is specific for a naturally occurring polynucleotide encoding a serine racemase protein. In particular preferred embodiments, the polynucleotides of this aspect are useful as probes for the specific detection of the presence of a polynucleotide encoding a serine racemase protein. In other particular embodiments, the polynucleotides of this aspect are useful as primers for use in nucleic acid amplification based assays for the specific detection of the presence of a polynucleotide encoding a serine racemase protein. In preferred embodiments, the polynucleotides of this aspect can have additional components including, but not limited to, compounds, isotopes, proteins or sequences for the detection of the probe or primer.

An aspect of this invention is an expression vector including a polynucleotide encoding a serine racemase protein, or a complementary sequence, and regulatory regions. In a particular embodiment the encoded protein has a sequence corresponding to SEQ ID NO:2. In particular embodiments, the vector can have any of a variety of regulatory regions known and used in the art as appropriate for the types of host cells the vector can be used in. In a most preferred embodiment, the vector has regulatory regions appropriate for the expression of the encoded protein in human host cells. In other embodiments, the vector has regulatory regions appropriate for expression of the encoded protein in bacteria, cyanobacteria, actinomycetes or a variety of eukaryotes including yeasts and insect cells. In some preferred embodiments the regulatory regions provide for inducible expression while in other preferred embodiments the regulatory regions provide for constitutive expression. Finally, according to this aspect, the expression vector can be derived from a plasmid, phage, virus, artificial chromosome or a combination thereof.

An aspect of this invention is host cell comprising an expression vector that includes a polynucleotide encoding a serine racemase polypeptide, or a complementary sequence, and appropriate regulatory regions. In a particular embodiment the polypeptide encoded by the vector has an amino acid sequence corresponding to SEQ ID NO:2. In preferred embodiments, the host cell is a eukaryote, yeast, insect cell, gram-positive bacterium, cyanobacterium or actinomycete. In a most preferred embodiment, the host cell is a human cell.

An aspect of this invention is a process for expressing a serine racemase protein in a host cell. In this aspect a host cell is transformed or transfected with an expression vector including a polynucleotide encoding a serine racemase protein, or a complementary sequence. According to this aspect, the host cell is cultured under conditions conducive to the expression of the encoded serine racemase protein. In particular embodiments the expression is inducible or constitutive. In a particular embodiment the encoded protein has a sequence corresponding to SEQ ID NO:2.

An aspect of this invention is a recombinant serine racemase polypeptide having an amino acid sequence of SEQ ID NO:2 or the equivalent sequence of a naturally occurring mutant or polymorphic form of the protein.

An aspect of this invention is a method of determining whether a candidate compound can alter the activity of a serine racemase polypeptide. According to this aspect a polynucleotide encoding the polypeptide is used to construct an expression vector appropriate for a particular host cell. The host cell is transformed or transfected with the expression vector and cultured under conditions conducive to the expression of the serine racemase polypeptide. Cells are optionally disrupted and, optionally, membranes are collected by centrifugation. The serine racemase may be purified if desired or cell extracts can be used directly. The cells, cell extracts, membranes, or serine racemase polypeptide purified from the cells are contacted with the candidate compounds. Finally, one measures the activity of the serine racemase polypeptide in the presence of the candidate. If the activity is lower relative to the activity of the enzyme in the absence of the candidate, then the candidate is an inhibitor of the serine racemase polypeptide. In preferred embodiments, the polynucleotide encodes a protein having an amino acid sequence of SEQ ID NO:2 or a naturally occurring mutant of polymorphic form thereof. In other preferred embodiments, the polynucleotide has the sequence of SEQ ID NO:1. In particular embodiments, the relative activity of serine racemase is determined by comparing the activity of the serine racemase to a control. In some embodiments, the host cell is contacted with the candidate and activity of serine racemase protein is determined by measuring a cell phenotype that is dependent upon serine racemase function, e.g., activation of an NMDA receptor. According to this aspect of the invention, the relative activity can be determined by comparison to a previously measured or expected activity value for the serine racemase activity under the conditions. However, in preferred embodiments, the relative activity is determined by measuring the activity of the serine racemase in a control sample that was not contacted with a candidate compound. In particular embodiments, the host cell is a mammalian cell and the protein inhibited is the recombinant serine racemase produced by the mammalian cell.

By “about” it is meant within 10% to 20% greater or lesser than particularly stated.

As used herein an “agonist” is a compound or molecule that interacts with and stimulates an activity of serine racemase.

As used herein an “antagonist” is a compound that interacts with serine racemase and interferes with the activity of serine racemase.

As used herein an “inhibitor” is a compound that interacts with and inhibits or prevents serine racemase from catalyzing the racemization of serine by serine racemase.

As used herein a “modulator” is a compound that interacts with an aspect of cellular biochemistry to effect an increase or decrease in the amount of a polypeptide of serine racemase present in, at the surface or in the periplasm of a cell, or in the surrounding serum or media. The change in amount of the serine racemase polypeptide can be mediated by the effect of a modulator on the expression of the protein, e.g., the transcription, translation, post-translational processing, translocation or folding of the protein, or by affecting a component(s) of cellular biochemistry that directly or indirectly participates in the expression of the protein. Alternatively, a modulator can act by accelerating or decelerating the turnover of the protein either by direct interaction with the protein or by interacting with another component(s) of cellular biochemistry which directly or indirectly effects the change.

An aspect of this invention is a non-human transgenic animal useful for the study of the tissue and temporal specific expression or activity of the serine racemase gene in an animal. The animal is also useful for studying the ability of a variety of compounds to act as agonists, antagonists or inhibitors of serine racemase activity or expression in vivo or, by providing cells for culture or assays, in vitro. In an embodiment of this aspect of the invention, the animal lacks a functional endogenous serine racemase gene. In another embodiment, the animal expresses a non-native serine racemase gene in the absence of the expression of a endogenous gene. In particular embodiments the non-human animal is a mouse. In further embodiments the non-native serine racemase gene is a wild-type human serine racemase gene or a mutant serine racemase gene.

All of the references cited herein are incorporated by reference in their entirety as background material.

### DETAILED DESCRIPTION OF THE INVENTION

The present invention provides polynucleotides and polypeptides of a human serine racemase, referred to herein as serine racemase. The polynucleotides and polypeptides are used to further provide expression vectors, host cells comprising the vectors, probes and primers, antibodies against the serine racemase protein and polypeptides thereof, assays for the presence or expression of serine racemase and assays for the identification of compounds that interact with serine racemase.

L-serine is an amino acid found in proteins. D-serine is an amino acid not typically incorporated in proteins, but nevertheless is found in limited distribution in the human body, particularly in the tissues of the nervous system. It is believed that D-serine is a ligand of NMDA receptor and is necessary for activation of NMDA receptors. D-Serine and L-serine are interconvertible by serine racemase. Therefore, it is believed that altering the activity of serine racemase is a means of altering the activation of NMDA receptors.

The present invention provides a cDNA encoding a human serine racemase enzyme was cloned using an approach that combined searching the EST database and DNA sequencing. The sequence of a full-length cDNA predicts an open reading frame of 1023 nucleotides encoding a protein of 341 amino acids for this serine racemase. The predicted protein shows 89% identity with the mouse serine racemase reported by Wolosker et al., 1999. Northern blot analysis of mRNA expression for this human enzyme demonstrated that it is expressed in brain, heart, skeletal muscle, kidney and liver. The human serine racemase gene was been mapped to chromosome 17pl3 by using GENEBRIDGE 4 Radiation Hybrid Panel and Stanford G3 Radiation hybrid Panel.

Drugs that act on the NMDA receptor glycine site for D-serine are currently being developed (Danysz and Parsons, 1998). The indicated therapeutic applications include treatments for stroke, depression and chronic pain. The discovery of human serine racemase provides another therapeutic approach to address disease states. D-serine is reported to be an endogenous activator for the NMDA receptor (glycine site) and the level of D-serine is changed during pathological conditions, such as major depression (Altamura et al., 1995), seizures (Ronneengstrom, 1992), and ischemia (Hirai and Okada, 1993). Therefore, modulation of serine racemase activity is a reasonable approach to address these disease states.

The key role of NMDA receptors in chronic pain state and hyperalgesia is well documented (Dickenson, 1990; Coderre, 1993). However, NMDA receptor blockers have two potentially serious side effects—neurodegenerative changes in the cingulate/retrosplenial cortex and psychotomimetic-like effects. Recent findings suggest that D-serine also plays an important role in hyperalgesia and pain. Jun et al., (1998) and Carlton et al., (1998) found that D-serine reversed the effects of gabapentin antihyperalgesic activity. Intrathecally administered D-serine potentiated the nociceptive responses of multireceptive spinal neurons to coloretal distension (Kolhekar and Gebhart, 1996). Therefore, an inhibitor of serine racemase which decreases D-serine concentration and decreases the activation at the glycine site might block the development of chronic pain state at doses causing few side effects. The combination of an inhibitor of serine racemase and other NMDA receptor antagonists might be a better and more efficient treatment than either treatment alone.

Activation of NMDA receptors following the massive release of glutamate seen after a stroke is thought to be responsible for the neural damage associated with this neuropathic event. Kanthan et al., (1995) reported that extracellular concentrations of serine, glutamine and glycine were dramatically increased in the simulated ischemic model of the temporal lobe of the human brain, as monitored by in vivo microdialysis. Therefore, inhibition of serine racemase provides a therapeutic target for NMDA-mediated stroke pathology, as well as neurodegenerative diseases in which glutamate excitotoxicity plays a pathophysiologic role.

High affinity NMDA channel blockers, such as PCP, mimic both the positive and negative symptoms of schizophrenia in humans (Javitt and Zukin, 1991). Moreover, supplementation with D-serine revealed significant improvements in positive, negative and cognitive symptoms of schizophrenic patients (Tsai, et al., 1998). Therefore, the pathophysiology of schizophrenia may be linked to hypofunction of the NMDA receptor, and an agonist of serine racemase might be useful for the treatment of schizophrenia.

Spinocerebellar atxia is one of the most common neurological disorders. However, few compounds provide effective treatment of this disorder. Saigoh et al., (1998) recently found that intraperitoneal administration of D-serine ethylester increased the extracellular content of endogenous D-serine in the mouse cerebellum and reduced the falling index of mice that exhibit cytosine arabinoside-induced ataxia. Therefore, an agonist of serine racemase may be useful in the treatment of spinocerebellar atxia.

Polynucleotides Polynucleotides useful in the present invention include those described herein and those that one of skill in the art will be able to derive therefrom following the teachings of this specification. A preferred aspect of the present invention is a recombinant polynucleotide encoding a human serine racemase polypeptide. One preferred embodiment is a nucleic acid having the sequence disclosed in SEQ ID NO:1 and disclosed as follows:

| | |
|---|---|
| ATGTGTGCTC AGTATTGCAT CTCCTTTGCT GATGTTGAAA AAGCTCATAT | (SEQ ID NO:1) |
| CAACATTCGA GATTCTATCC ACCTCACACC AGTGCTAACA AGCTCCATTT | |
| TGAATCAACT AACAGGGCGC AATCTTTTCT TCAAATGTGA ACTCTTCCAG | |
| AAAACAGGAT CTTTTAAGAT TCGTGGTGCT CTCAATGCCG TCAGAAGCTT | |
| GGTTCCTGAT GCTTTAGAAA GGAAGCCGAA AGCTGTTGTT ACTCACAGCA | |
| GTGGAAACCA TGGCCAGGCT CTCACCTATG CTGCCAAATT GGAAGGAATT | |
| CCTGCTTATA TTGTGGTGCC CCAGACAGCT CCAGACTGTA AAAAACTTGC | |
| AATACAAGCC TACGGAGCGT CAATTGTATA CTGTGAACCT AGTGATGAGT | |
| CCAGAGAAAA TGTTGCAAAA AGAGTTACAG AAGAAACAGA AGGCATCATG | |
| GTACATCCCA ACCAGGAGCC TGCAGTGATA GCTGGACAAG GGACAATTGC | |
| CCTGGAAGTG CTGAACCAGG TTCCTTTGGT GGATGCACTG GTGGTACCTG | |
| TAGGTGGAGG AGGAATGCTT GCTGGAATAG CAATTACAGT TAAGGCTCTG | |
| AAACCTAGTG TGAAGGTATA TGCTGCTGAA CCCTCAAATG CAGATGACTG | |
| CTACCAGTCC AAGCTGAAGG GGAAACTGAT GCCCAATCTT TATCCTCCAG | |
| AAACCATAGC AGATGGTGTC AAATCCAGCA TTGGCTTGAA CACCTGGCCT | |
| ATTATCAGGG ACCTTGTGGA TGATATCTTC ACTGTCACAG AGGATGAAAT | |
| TAAGTGTGCA ACCCAGCTGG TGTGGGAGAG GATGAAACTA CTCATTGAAC | |
| CTACAGCTGG TGTTGGAGTG GCTGCTGTGC TGTCTCAACA TTTTCAAACT | |
| GTTTCCCCAG AAGTAAAGAA CATTTGTATT GTGCTCAGTG GTGGAAATGT | |
| AGACTTAACC TCCTCCATAA CTTGGGTGAA GCAGGCTGAA AGGCCAGCTT | |
| CTTATCAGTC TGTTTCTGTT TAA | |

A particularly preferred embodiment is a polynucleotide comprising the entire coding sequence of serine racemase of SEQ ID NO:1.

The isolated nucleic acid molecules of the present invention can include a ribonucleic or deoxyribonucleic acid molecule, which can be single (coding or noncoding strand) or double stranded, as well as synthetic nucleic acid, such as a synthesized, single stranded polynucleotide.

The present invention also relates to recombinant vectors and recombinant hosts, both prokaryotic and eukaryotic, which contain the recombinant nucleic acid molecules disclosed throughout this specification.

As used herein a “polynucleotide” is a nucleic acid of more than one nucleotide. A polynucleotide can be made up of multiple polynucleotide units that are referred to by description of the unit. For example, a polynucleotide can comprise within its bounds a polynucleotide(s) having a coding sequence(s), a polynucleotide(s) that is a regulatory region(s) and/or other polynucleotide units commonly used in the art.

An “expression vector” is a polynucleotide having regulatory regions operably linked to a coding region such that, when in a host cell, the regulatory regions can direct the expression of the coding sequence. The use of expression vectors is well known in the art. Expression vectors can be used in a variety of host cells and, therefore, the regulatory regions are preferably chosen as appropriate for the particular host cell.

A “regulatory region” is a polynucleotide that can promote or enhance the initiation or termination of transcription or translation of a coding sequence. A regulatory region includes a sequence that is recognized by the RNA polymerase, ribosome, or associated transcription or translation initiation or termination factors of a host cell. Regulatory regions that direct the initiation of transcription or translation can direct constitutive or inducible expression of a coding sequence.

Polynucleotides of this invention contain full length or partial length sequences of the serine racemase gene sequences disclosed herein. Polynucleotides of this invention can be single or double stranded. If single stranded, the polynucleotides can be a coding, “sense,” strand or a complementary, “antisense,” strand. Antisense strands can be useful as modulators of the gene by interacting with RNA encoding the serine racemase protein. Antisense strands are preferably less than full length strands having sequences unique or specific for RNA encoding the protein.

The polynucleotides can include deoxyribonucleotides, ribonucleotides or mixtures of both. The polynucleotides can be produced by cells, in cell-free biochemical reactions or through chemical synthesis. Non-natural or modified nucleotides, including without limitation inosine, methyl-cytosine, deaza-guanosine, etc., can be present. Natural phosphodiester internucleotide linkages can be appropriate. However, polynucleotides can have non-natural linkages between the nucleotides. Non-natural linkages are well known in the art and include, without limitation, methylphosphonates, phosphorothioates, phosphorodithionates, phosphoroamidites and phosphate ester linkages. Dephospho-linkages are also known, as bridges between nucleotides. Examples of these include siloxane, carbonate, carboxymethyl ester, acetamidate, carbamate, and thioether bridges. “Plastic DNA,” having, for example, N-vinyl, methacryloxyethyl, methacrylamide or ethyleneimine internucleotide linkages, can be used. “Peptide Nucleic Acid” (PNA) is also useful and resists degradation by nucleases. These linkages can be mixed in a polynucleotide.

As used herein, “purified” and “isolated” are utilized interchangeably to stand for the proposition that the polynucleotide, protein and polypeptide, or respective fragments thereof in question have been removed from the in vivo environment so that they exist in a form or purity not found in nature. Purified or isolated nucleic acid molecules can be manipulated by the skilled artisan, such as but not limited to sequencing, restriction digestion, site-directed mutagenesis, and subcloning into expression vectors for a nucleic acid fragment as well as obtaining the wholly or partially purified protein or protein fragment so as to afford the opportunity to generate polyclonal antibodies, monoclonal antibodies, or perform amino acid sequencing or peptide digestion. Therefore, the nucleic acids claimed herein can be present in whole cells or in cell lysates or in a partially or substantially purified form. It is preferred that the molecule be present at a concentration at least about five-fold to ten-fold higher than that found in nature. A polynucleotide is considered substantially pure if it is obtained purified from cellular components by standard methods at a concentration of at least about 100-fold higher than that found in nature. A polynucleotide is considered essentially pure if it is obtained at a concentration of at least about 1000-fold higher than that found in nature. We most prefer polynucleotides that have been purified to homogeneity, that is, at least 10,000-100,000 fold. A chemically synthesized nucleic acid sequence is considered to be substantially purified when purified from its chemical precursors by the standards stated above.

The term “recombinant” is used to denote those polynucleotide preparations, constructs, expression vectors, integrated sequences and cell lines containing the same which are made by the hand of man.

Included in the present invention are assays that employ further novel polynucleotides that hybridize to serine racemase sequences under stringent conditions. By way of example, and not limitation, a procedure using conditions of high stringency is as follows: Prehybridization of filters containing DNA is carried out for 2 hr. to overnight at 65° C. in buffer composed of 6×SSC, 5 × Denhardt's solution, and 100 μg/ml denatured salmon sperm DNA. Filters are hybridized for 12 to 48 hrs at 65° C. in prehybridization mixture containing 100 μg/ml denatured salmon sperm DNA and 5-20×10⁶ cpm of ³²P-labeled probe. Washing of filters is done at 37° C. for 1 hr in a solution containing 2×SSC, 0.1% SDS. This is followed by a wash in 0.1×SSC, 0.1% SDS at 50° C. for 45 min. before autoradiography.

Other procedures using conditions of high stringency would include either a hybridization step carried out in 5×SSC, 5× Denhardt's solution, 50% formamide at 42° C. for 12 to 48 hours or a washing step carried out in 0.2× SSPE, 0.2% SDS at 65° C. for 30 to 60 minutes.

Reagents mentioned in the foregoing procedures for carrying out high stringency hybridization are well known in the art. Details of the composition of these reagents can be found in, e.g., Sambrook, et al., 1989, Molecular Cloning: A Laboratory Manual, second edition, Cold Spring Harbor Laboratory Press. In addition to the foregoing, other conditions of high stringency which may be used are well known in the art. “Identity” is a measure of the identity of nucleotide sequences or amino acid sequences. In general, the sequences are aligned so that the highest order match is obtained. “Identity” per se has an art-recognized meaning and can be calculated using published techniques. See, e.g.,: (COMPUTATIONAL MOLECULAR BIOLOGY, Lesk, A. M., ed. Oxford University Press, New York, 1988; BIOCOMPUTING: INFORMATICS AND GENOME PROJECTS, Smith, D. W., ed., Academic Press, New York, 1993; COMPUTER ANALYSIS OF SEQUENCE DATA, PART I, Griffin, A. M., and Griffin, H. G., eds. Humana Press, New Jersey, 1994; SEQUENCE ANALYSIS IN MOLECULAR BIOLOGY, von Heinje, G., Academic Press, 1987; and SEQUENCE ANALYSIS PRIMER, Gribskov, M. and Devereux, J., eds., M Stockton Press, New York, 1991). While there exist a number of methods to measure identity between two polynucleotide or polypeptide sequences, the term “identity” is well known to skilled artisans (Carillo, H., and Lipton, D., SIAM J Applied Math (1988) 48:1073). Methods commonly employed to determine identity or similarity between two sequences include, but are not limited to, those disclosed in Guide to Huge Computers, Martin J. Bishop, ed., Academic Press, San Diego, 1994, and Carillo, H., and Lipton, D., SIAM J Applied Math (1988) 48:1073. Methods to determine identity and similarity are codified in computer programs. Preferred computer program methods to determine identity and similarity between two sequences include, but are not limited to, GCG program package (Devereux, J. et a., Nucleic Acids Research (1984) 12(1):387), BLAST?, BLASTN, FASTA (Atschul, S. F. et al., J Molec Biol (1990) 215:403).

As an illustration, by a polynucleotide having a nucleotide sequence having at least, for example, 95% “identity” to a reference nucleotide sequence of SEQ ID NO:1 is intended that the nucleotide sequence of the polynucleotide is identical to the reference sequence except that the polynucleotide sequence may include up to five point mutations per each 100 nucleotides of the reference nucleotide sequence of SEQ ID NO:1. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5 or 3 terminal positions of the reference nucleotide sequence or anywhere between those terminal positions, interspersed either individually among nucleotides in the reference sequence or in one or more contiguous groups within the reference sequence.

Similarly, by a polypeptide having an amino acid sequence having at least, for example, 95% identity to a reference amino acid sequence of SEQ ID NO:2 is intended that the amino acid sequence of the polypeptide is identical to the reference sequence except that the polypeptide sequence may include up to five amino acid alterations per each 100 amino acids of the reference amino acid of SEQ ID NO:2. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence of anywhere between those terminal positions, interspersed either individually among residues in the reference sequence or in one or more contiguous groups within the reference sequence.

Polypeptides

A preferred aspect of the present invention is a substantially purified form of the human serine racemase protein. A preferred embodiment is a protein that has the amino acid sequence which is disclosed in SEQ D) NO:2 and disclosed in single letter code as follows:

(SEQ ID NO:2)

MCAQYCISFADVEKAHINIRDSIHLTPVLTSSILNQLTGRNLFFKCELFQ
KTGSFKIRGALNAVRSLVPDALERKPKAVVTHSSGNHGQALTYAAKLEGI
PAYIVVPQTAPDCKKLAIQAYGASIVYCEPSDESRENVAKRVTEETEGIM
VHPNQEPAVIAGQGTIALEVLNQVPLVDALVVPVGGGGMLAGIAITVKAL
KPSVKVYAAEPSNADDCYQSKLKGKLMPNLYPPETIADGVKSSIGLNTWP
IIRDLVDDIFTVTEDEIKCATQLVWERMKLLIEPTAGVGVAAVLSQHFQT
VSPEVKNICIVLSGGNVDLTSSITWVKQAERPASYQSVSV

The underlined sequences, which were searched by using BLOCKS bioinformatic software, have a consensus sequence for pyridoxal 5′ phosphate (BLOCKS accession number BL00165A and BL00165B).

The present invention also relates to biologically active fragments and mutant or polymorphic forms of the serine racemase polypeptide sequence set forth as SEQ ID NO:2, including but not limited to amino acid substitutions, deletions, additions, amino terminal truncations and carboxy-terminal truncations such that these mutations provide for proteins or protein fragments of diagnostic, therapeutic or prophylactic use and would be useful for screening for modulators, and/or inhibitors of serine racemase function.

Using the disclosure of polynucleotide and polypeptide sequences provided herein to isolate polynucleotides encoding naturally occurring forms of serine racemase, one of skill in the art can determine whether such naturally occurring forms are mutant or polymorphic forms of serine racemase by sequence comparison. One can further determine whether the encoded protein, or fragments of any serine racemase protein, is biologically active by routine testing of the protein of fragment in a in vitro or in vivo assay for the biological activity of the serine racemase protein. For example, one can express N-terminal or C-terminal truncations, or internal additions or deletions, in host cells and test for their ability to catalyze the racemization of serine.

It is known that there is a substantial amount of redundancy in the various codons which code for specific amino acids. Therefore, this invention is also directed to those DNA sequences that encode RNA comprising alternative codons which code for the eventual translation of the identical amino acid.

Therefore, the present invention discloses codon redundancy which can result in different DNA molecules encoding an identical protein. For purposes of this specification, a sequence bearing one or more replaced codons will be defined as a degenerate variation. Also included within the scope of this invention are mutations either in the DNA sequence or the translated protein which do not substantially alter the ultimate physical properties of the expressed protein. For example, substitution of valine for leucine, arginine for lysine, or asparagine for glutamine may not cause a change in functionality of the polypeptide. However, any given change can be examined for any effect on biological function by simply assaying for the ability to catalyze the racemization of serine as compared to an unaltered serine racemase protein.

It is known that DNA sequences coding for a peptide can be altered so as to code for a peptide having properties that are different than those of the naturally occurring peptide. Methods of altering the DNA sequences include but are not limited to site directed mutagenesis. Examples of altered properties include but are not limited to changes in the affinity of an enzyme for a substrate.

As used herein in reference to a serine racemase gene or encoded protein, a “polymorphic” serine racemase is a serine racemase that is naturally found in the population of animals at large. Typically, the genes for polymorphs of serine racemase can be detected by high stringency hybridization using the serine racemase gene as a probe. A polymorphic form of serine racemase can be encoded by a nucleotide sequence different from the particular serine racemase gene disclosed herein as SEQ ID NO:1. However, because of silent mutations, a polymorphic serine racemase gene can encode the same or different amino acid sequence as that disclosed herein. Further, some polymorphic forms serine racemase will exhibit biological characteristics that distinguish the form from wild-type serine racemase activity, in which case the polymorphic form is also a mutant.

The invention includes a serine racemase polypeptide which has been modified by deletion, addition, modification or substitution of one or more amino acid residues in the wild-type enzyme. It encompasses allelic and polymorphic variants, and fusion proteins which comprise all or a significant part of a polypeptide, e.g., covalently linked via a side-chain group or terminal residue to a different protein, polypeptide or moiety (fusion partner).

Some amino acid substitutions are preferably “conservative”, with residues replaced with physicochemically similar residues, such as Gly/Ala, Asp/Glu, Val/Ile/Leu, Lys/Arg, Asn/Gln and Phe/Trp/Tyr. Analogs of enzymes having such conservative substitutions typically retain substantial enzymatic activity. Other analogs, which have non-conservative substitutions such as Asn/Glu, Val/Tyr and His/Glu, may substantially lack enzymatic activity. Nevertheless, such analogs are useful because they can be used as antigens to elicit production of antibodies in an immunologically competent host. Because these analogs retain many of the epitopes (antigenic determinants) of the wild-type enzymes from which they are derived, many antibodies produced against them can also bind to the active-conformation or denatured wild-type enzymes. Accordingly, the antibodies can be used, e.g., for the immunopurification or immunoassay of the wild-type enzymes.

Whether a particular analog exhibits serine racemase activity can be determined by routine experimentation as described herein.

Some analogs are truncated variants in which residues have been successively deleted from the amino- and/or carboxyl-termini, while substantially retaining the characteristic serine racemase activity.

Modifications of amino acid residues may include but are not limited to aliphatic esters or amides of the carboxyl terminus or of residues containing carboxyl side chains, O-acyl derivatives of hydroxyl group-containing residues, and N-acyl derivatives of the amino-terminal amino acid or amino-group containing residues, e.g., lysine or arginine.

This invention also encompasses physical variants having substantial amino acid sequence homology with the amino acid sequences of the serine racemase polypeptide sometimes referred to as analogs. In this invention, amino acid sequence homology, or sequence identity, is determined by optimizing residue matches and, if necessary, by introducing gaps as required. Homologous amino acid sequences are typically intended to include natural allelic, polymorphic and interspecies variations in each respective sequence.

Typical homologous proteins or peptides will have from 25-100% homology (if gaps can be introduced) to 50-100% homology (if conservative substitutions are included), with the amino acid sequence of the serine racemase. Primate species serine racemases are of particular interest.

Observed homologies will typically be at least about 35%, preferably at least about 50%, more preferably at least about 75%, and most preferably at least about 85% or more. See Needleham et al., J. Mol. Biol. 48:443-453 (1970); Sankoff et al. in Time Warps, String Edits, and Macromolecules: The Theory and Practice of Sequence Comparison, 1983, Addison-Wesley, Reading, Mass.; and software packages from IntelliGenetics, Mountain View, Calif., and the University of Wisconsin Genetics Computer Group, Madison, Wis. In particularly preferred embodiments of the present invention, the serine racemase polypeptide has at least 90%, 91%, 92%, 93%, 94%, 95%, 96% 97%, 98%, 99% or greater homology as compared to the serine racemase of SEQ ID NO:2.

In some preferred embodiments of this invention, one can start with the murine serine racemase sequence known in the art and, using the serine racemase polypeptide of SEQ ID NO:2 as a guide, design a serine racemase polypeptide which is more like the human sequence. For example, one can determine locations in the murine sequence that are different from the human sequence and, at one or more of those positions, change the amino acid from that occurring in the murine sequence to that occurring in the human sequence. Alternatively, one can state with the human sequence and make changes to the amino acids appearing in the murine sequence. In some embodiments hereunder the resulting serine racemase polypeptide has at least 90%, 91%, 92%, 93%, 94%, 95%, 96% 97%, 98%, 99% or greater homology as compared to the serine racemase of SEQ ID NO:2. Because of the large number of different permutations of amino acid sequences that can be designed by comparing the murine and human sequences and making appropriate changes as taught herein, we refer to the different subsets of polypeptides by their percent (%) homology whereby the 90% homologous group has the largest number of members and the 99% homologous group has the smallest number of members.

Glycosylation variants include, e.g., analogs made by modifying glycosylation patterns during synthesis and processing in various alternative eukaryotic host expression systems, or during further processing steps. Particularly preferred methods for producing glycosylation modifications include exposing the polypeptide to glycosylating enzymes derived from cells which normally carry out such processing, such as mammalian glycosylation enzymes. Alternatively, deglycosylation enzymes can be used to remove carbohydrates attached during production in eukaryotic expression systems.

Other analogs are serine racemase polypeptides containing modifications, such as incorporation of unnatural amino acid residues, or phosphorylated amino acid residues such as phosphotyrosine, phosphoserine or phosphothreonine residues. Other potential modifications include sulfonation, biotinylation, or the addition of other moieties, particularly those which have molecular shapes similar to phosphate groups.

Analogs of the human serine racemases can be prepared by chemical synthesis or by using site-directed mutagenesis (Gillman et al., Gene 8:81 (1979); Roberts et al., Nature 328:731 (1987) or Innis (Ed.), 1990, PCR Protocols: A Guide to Methods and Applications, Academic Press, New York, N.Y.) or the polymerase chain reaction method (PCR; Saiki et al., Science 239:487 (1988)), as exemplified by Daugherty et al. (Nucleic Acids Res. 19:2471 (1991)) to modify nucleic acids encoding the complete enzyme. Adding epitope tags for purification or detection of recombinant products is envisioned.

A protein or fragment thereof is considered purified or isolated when it is obtained at least partially free from it's natural environment in a composition or purity not found in nature. It is preferred that the molecule be present at a concentration at least about five-fold to ten-fold higher than that found in nature. A protein or fragment thereof is considered substantially pure if it is obtained at a concentration of at least about 100-fold higher than that found in nature. A protein or fragment thereof is considered essentially pure if it is obtained at a concentration of at least about 1000-fold higher than that found in nature. It is most prefer proteins that have been purified to homogeneity, that is, at least 10,000-100,000 fold.

The term “recombinant” with respect to a polypeptide of the present invention refers only to polypeptides that are made by recombinant processes, expressed by recombinant host cells or purified from natural cells as described herein or as known in the art. Preparations having partially purified serine racemase polypeptide are meant to be within the scope of the term “recombinant.”

Expression of Serine Racemase

A variety of expression vectors can be used to express recombinant serine racemase polypeptide in host cells. Expression vectors are defined herein as nucleic acid sequences that include regulatory sequences for the transcription of cloned DNA and the translation of their mRNAs in an appropriate host. Such vectors can be used to express a genes in a variety of hosts such as yeast, bacteria, bluegreen algae, plant cells, insect cells and animal cells. Specifically designed vectors allow the shuttling of genes between hosts such as bacteria-yeast or bacteria-animal cells. An appropriately constructed expression vector should contain: an origin of replication for autonomous replication in host cells, selectable markers, a limited number of useful restriction enzyme sites, a potential for high copy number, and regulatory sequences. A promoter is defined as a regulatory sequence that directs RNA polymerase to bind to DNA and initiate RNA synthesis. A strong promoter is one which causes mRNAs to be initiated at high frequency. Expression vectors can include, but are not limited to, cloning vectors, modified cloning vectors, specifically designed plasmids or viruses.

In particular, a variety of bacterial expression vectors can be used to express recombinant serine racemase in bacterial cells. Commercially available bacterial expression vectors which are suitable for recombinant serine racemase expression include, but are not limited to pQE (QIAGEN), pET11a or pET15b (NOVAGEN), lambda gt11 (INVITROGEN), and pKK223-3 (PHARMACIA).

Alternatively, one can express serine racemase DNA in cell-free transcription-translation systems, or serine racemase RNA in cell-free translation systems. Cell-free synthesis of serine racemase polypeptide can be in batch or continuous formats known in the art.

One can also synthesize serine racemase chemically, although this method is not preferred.

A variety of host cells can be employed with expression vectors to synthesize serine racemase protein. These can include E. coli, Bacillus, and Salmonella. Insect and yeast cells can also be appropriate. However, the most preferred host cell is a human host cell.

Following expression of serine racemase in a host cell, serine racemase polypeptides can be recovered. Several protein purification procedures are available and suitable for use. Serine racemase protein and polypeptides can be purified from cell lysates and extracts, or from culture medium, by various combinations of, or individual application of methods including detergent solubilization, ultrafiltration, acid extraction, alcohol precipitation, salt fractionation, ionic exchange chromatography, phosphocellulose chromatography, lecithin chromatography, affinity (e.g., antibody or His-Ni) chromatography, size exclusion chromatography, hydroxylapatite adsorption chromatography and chromatography based on hydrophobic or hydrophilic interactions. In some instances, protein denaturation and refolding steps can be employed. High performance liquid chromatography (HPLC) and reversed phase HPLC can also be useful. Dialysis can be used to adjust the final buffer composition.

The serine racemase protein itself is useful in assays to identify compounds that alter the activity of the enzyme—including compounds that inhibit or stimulate the activity of the enzyme. The serine racemase protein is also useful for the generation of antibodies against the protein, structural studies of the protein, and structure/function relationships of the protein.

Modulators, Agonist, Antagonists and Inhibitors of Serine Racemase

The present invention is also directed to methods for screening for compounds which modulate the expression of, stimulate or inhibit the activity of a serine racemase protein. Compounds which modulate, stimulate or inhibit serine racemase can be DNA, RNA, peptides, proteins, or non-proteinaceous organic or inorganic compounds or other types of molecules. Compounds that modulate the expression of DNA or RNA encoding serine racemase or are agonists, antagonists or inhibitors of the biological function of serine racemase can be detected by a variety of assays. The assay can be a simple qualitative “yes/no” assay to determine whether there is a change in expression or activity. The assay can be made quantitative by comparing the expression or activity of a test sample with the level or degree of expression or activity in a standard sample, e.g., compared to a control. A compound that is a modulator can be detected by measuring the amount of the mRNA and/or serine racemase produced in the presence of the compound. A compound that is an agonist, antagonist or inhibitor can be detected by measuring the specific activity of the serine racemase protein in the presence and absence of the compound. Control assays are run under the same conditions as test assays except that the test compound is omitted from the assay.

The proteins, DNA molecules, RNA molecules and antibodies lend themselves to the formulation of kits suitable for the detection and analysis of serine racemase. Such a kit would comprise a compartmentalized carrier suitable to hold in close confinement at least one container. The carrier would further comprise reagents such as recombinant serine racemase or anti-serine racemase antibodies suitable for detecting serine racemase. The carrier can also contain a means for detection such as labeled antigen or enzyme substrates or the like.

Assays

Assays of the present invention can be designed in many formats generally known in the art of screening compounds for biological activity or for binding to enzymes. Assays of the present invention can advantageously exploit the activity of serine racemase in converting L-serine to D-serine. D-serine can be detected directly or a secondary signal can be detected, e.g., the D-serine induced activation of a NMDA receptor.

The present invention includes methods of identifying compounds that specifically interact with serine racemase polypeptides. Compounds that interact with the enzyme can stimulate or inhibit the activity of serine racemase. The specificity of binding of compounds having affinity for serine racemase can be shown by measuring the affinity of the compounds to serine racemase isolated from recombinant cells expressing a serine racemase polypeptide. Expression of serine racemase polypeptides and screening for compounds that bind to serine racemase or that inhibit the conversion of L-serine to D-serine, provides an effective method for the rapid selection of compounds with affinity for serine racemase. The L-serine can be labeled by means known in the art, including a radiolabel, and thereafter can be used to follow the conversion of the labeled L-serine to D-serine in assays of serine racemase activity.

If one desires to produce an analog, fragment of the serine racemase or mutant, polymorphic or allelic variants of the serine racemase, one can test those polypeptides in the assays described below and compare the results to those obtained using an active serine racemase polypeptide of SEQ ID NO:2. In this manner one can easily assess the ability of the analog, fragment, mutant, polymorph or allelic variant to bind compounds, be activated by agonists or be inactivated or inhibited by antagonists of serine racemase.

Therefore, the present invention includes assays by which compounds that are serine racemase agonists, antagonists, and inhibitors may be identified. The assay methods of the present invention differ from those described in the art because the present assays incorporate at least one step wherein a serine racemase polypeptide of this invention is used in the assay.

General methods for identifying ligands, agonists and antagonists are well known in the art and can be adapted to identify agonists and antagonists of serine racemase. The order of steps in any given method can be varied or performed concurrently as will be recognized by those of skill in the art of assays. The following is a sampling of the variety of formats that can be used to conduct an assay of the present invention.

Accordingly, the present invention includes a method for determining whether a candidate compound is an agonist or an inhibitor of serine racemase, the method of which comprises:

(a) transfecting cells with an expression vector encoding a serine racemase polypeptide;

(b) allowing the transfected cells to grow for a time sufficient to allow serine racemase to be expressed in the cells;

(c) exposing portions of the cells to labeled L-serine in the presence and in the absence of the candidate compound;

(d) measuring the conversion of the labeled L-serine to D-serine in the portions of cells; and

(e) comparing the amount of conversion of L-serine to D-serine in the presence and the absence of the compound where a decrease in the amount of conversion of L-serine to D-serine in the presence of the compound indicates that the compound is an inhibitor of serine racemase whereas an increase in the conversion of L-serine to D-serine indicates that the compound is an agonist of serine racemase.

The conditions under which step (c) of the method is practiced are conditions that are typically used in the art for the study of protein-ligand interactions: e.g., physiological pH; salt conditions such as those represented by such commonly used buffers as PBS or in tissue culture media; a temperature of about 4° C. to about 45° C. In this step the L-serine and candidate compound can be applied to the cell sequentially or concurrently. It may be preferably that the compound is applied first or that the compound and L-serine are applied concurrently.

The above whole cell methods can be used in assays where one desires to assess whether a compound can traverse a cell membrane to interact with serine racemase. However, the above methods can be modified in that, rather than exposing the test cells to the candidate compound, extracts can be prepared from the cells and those extracts can be exposed to the compound. Such a modification utilizing extracts rather than cells is well known in the art. Particular methods of assaying are described in the Examples below.

Accordingly, the present invention provides a method of using the interaction of serine racemase and L-serine for determining whether a candidate compound is an agonist or inhibitor of a serine racemase polypeptide in extracts comprising:

(a) providing test cells by transfecting cells with an expression vector that directs the expression of serine racemase in the cells;

(b) preparing extracts containing serine racemase from the test cells;

(c) exposing the extracts to a candidate compound under conditions such that the ligand binds to the polypeptide in the extracts;

(d) measuring the amount of conversion of L-serine to D-serine in the extracts in the presence and the absence of the compound;

(e) comparing the amount of conversion of L-serine to D-serine in the presence and the absence of the compound where a decrease in the amount of conversion of L-serine to D-serine in the presence of the compound indicates that the compound is an inhibitor of serine racemase; whereas an increase in the conversion of L-serine to D-serine indicates that the compound is an agonist of serine racemase.

As a further modification of the above-described methods, RNA encoding serine racemase can be prepared as, e.g., by in vitro transcription using a plasmid containing serine racemase under the control of a bacteriophage T7 promoter, and the RNA can be microinjected into Xenopus oocytes in order to cause the expression of serine racemase in the oocytes. Compounds are then tested for binding to the serine racemase or inhibition of activity of serine racemase expressed in the oocytes. As in all assays of this invention, a step using a serine racemase polypeptide disclosed herein is incorporated into the assay.

Transgenic Animals

In reference to the transgenic animals of this invention, we refer to transgenes and genes. As used herein, a “transgene” is a genetic construct including a gene. The transgene is typically integrated into one or more chromosomes in the cells in an animal or its ancestor by methods known in the art. Once integrated, the transgene is carried in at least one place in the chromosomes of a transgenic animal. A gene is a nucleotide sequence that encodes a protein. The gene and/or transgene can also include genetic regulatory elements and/or structural elements known in the art.

The term “animal” is used herein to include all mammals, except humans. It also includes an individual animal in all stages of development, including embryonic and fetal stages. Preferably the animal is a rodent, and most preferably mouse or rat. A “transgenic animal” is an animal containing one or more cells bearing genetic information received, directly or indirectly, by deliberate genetic manipulation at a subcellular level, such as by microinjection or infection with recombinant virus. This introduced DNA molecule can be integrated within a chromosome, or it can be extra-chromosomally replicating DNA. Unless otherwise noted or understood from the context of the description of an animal, the term “transgenic animal” as used herein refers to a transgenic animal in which the genetic information was introduced into a germ line cell, thereby conferring the ability to transfer the information to offspring. If offspring in fact possess some or all of the genetic information, then they, too, are transgenic animals. The genetic information is typically provided in the form of a transgene carried by the transgenic animal.

The genetic information received by the non-human animal can be foreign to the species of animal to which the recipient belongs, or foreign only to the particular individual recipient. In the last case, the information can be altered or it can be expressed differently than the native gene. Alternatively, the altered or introduced gene can cause the native gene to become non-functional to produce a “knockout” animal.

As used herein, a “targeted gene” or “Knockout” (KO) transgene is a DNA sequence introduced into the germline of a non-human animal by way of human intervention, including but not limited to, the methods described herein. The targeted genes of the invention include nucleic acid sequences which are designed to specifically alter cognate endogenous alleles of the non-human animal.

An altered serine racemase gene should not fully encode the same protein endogenous to the host animal, and its expression product can be altered to a minor or great degree, or absent altogether. In cases where it is useful to express a non-native serine racemase protein in a transgenic animal in the absence of a endogenous serine racemase protein we prefer that the altered serine racemase gene induce a null, “knockout,” phenotype in the animal. However a more modestly modified serine racemase gene can also be useful and is within the scope of the present invention.

A type of target cell for transgene introduction is the embryonic stem cell (ES). ES cells can be obtained from pre-implantation embryos cultured in vivo and fused with embryos (M. J. Evans et al., Nature 292:154-156 (1981); Bradley et al., Nature 309:255-258 (1984); Gossler et al. Proc. Natl. Acad. Sci. USA 83:9065-9069 (1986); and Robertson et al., Nature 322:445448 (1986)). Transgenes can be efficiently introduced into the ES cells by a variety of standard techniques such as DNA transfection, microinjection, or by retrovirus-mediated transduction. The resultant transformed ES cells can thereafter be combined with blastocysts from a non-human animal. The introduced ES cells thereafter colonize the embryo and contribute to the germ line of the resulting chimeric animal (R. Jaenisch, Science 240: 1468-1474 (1988)). Animals are screened for those resulting in germline transformants. These are crossed to produce animals homozygous for the transgene.

Methods for evaluating the targeted recombination events as well as the resulting knockout mice are readily available and known in the art. Such methods include, but are not limited to DNA (Southern) hybridization to detect the targeted allele, polymerase chain reaction (PCR), polyacrylamide gel electrophoresis (PAGE) and Western blots to detect DNA, RNA and protein.

A particularly preferred embodiment of the present invention is a transgenic animal wherein the human serine racemase is expressed in the absence of the animal's endogenous serine racemase. Most preferably, the animal is a rat or a mouse wherein the endogenous serine racemase is knocked out and the human serine racemase is knocked-in. The phenotype of the animal is similar to a wild type phenotype because the human gene replaces the activity of the murine gene. However, the animal differs from wild-type in that the human serine racemase is detectable in the animal in the absence of a functional murine serine racemase.

This may have a therapeutic aim. The presence of a mutant, allele or variant sequence within cells of an organism, particularly when in place of a homologous endogenous sequence, may allow the organism to be used as a model in testing and/or studying the role of the serine racemase gene or substances which modulate activity of the encoded polypeptide and/or promoter in vivo or are otherwise indicated to be of therapeutic potential.

The Example below are included to describe certain aspects of the invention and do not define the scope of the invention. The protectable scope of the invention is limited only by the claims below.

### EXAMPLE 1

Identification of a Human Serine Racemase and cDNA Cloning.

The DNA sequence of mouse serine racemase was used to search the Genbank Human EST (Expressed Sequence Tag). The search resulted a human EST (GenBank accession number h73097) which contained partial human serine racemase sequence (353 bp at 5′ end). This human EST (h73097) was purchased from Research Genetics Inc. The clone was cultured on LB agar plate (Remel) containing 100 ug/ml ampicillin at 37° C. overnight. Five single colonies were picked and cultured in 5 ml LB media containing 50 ug/ml ampicillin at 37° C. for 16 hr. Plasmid DNA of this particular clone was isolated by using WIZARD PLUS Minipreps DNA Purification System (PROMEGA).

The purified DNA was sequenced with a universal T3 promoter primer, a T7 promoter primer and a M13/pUC reverse 23-base sequencing primer (GIBCO BRL). Sequencing was performed on an ABI PRISM 377 DNA sequencer (PERKIN ELMER). In addition, two internal primers were designed (forward primer: 5′-CTT GCA ATA CAA GCC TAC GGA GC-3′ (SEQ ID NO:3) and reverse primer: 5′-GTT CAA GCC AAT GCT GGA TTT GAC-3′ (SEQ ID NO:4)) and used for sequencing the internal region of this clone. The clone was sequenced through in both the 5′ and 3′ directions. The DNA sequence was assembled to generate the full-length sequence of the human serine racemase by using bioinformatic contig tools. The amino acid sequence of the serine racemase was deduced from the DNA sequence.

### EXAMPLE 2

Analysis of Expression of Human Serine Racemase.

A northern blot of poly(A+)-RNA isolated from human brain, heart, skeletal muscle, colon, thymus, spleen, kidney, liver, small intestine, placenta, lung, and peripheral blood leukocyte was purchased from CLONTECH (Palo Alto, Calif.). The probe of cDNA fragment (573 bp) from human serine racemase was labeled by using MULTIPRIME DNA labeling systems (AMERSHAM). The hybridization was carried out in 5×SSPE, 10 × Denhardt's solution, 50% formamide, 2% SDS, 20 ug/ml denatured salmon sperm DNA and 10⁸ cpm of ³²P-labeled probe at 42° C. for 18 hr. The membrane was washed stepwise in a solution containing 2×SSC, 0.05% SDS at 42° C. for 40 min, followed by 1 ×SSC, 0.05% SDS at 50° C. for 40 min. High stringency washes were carried out at 0.1 ×SSC, 0.05% SDS at 50° C. for 20 min. Then the membrane was detected by exposure of the blots to Kodak XAR X-ray film. Northern blot analysis of mRNA expression for human serine racemase demonstrated that the mRNA is expressed in human brain, heart, skeletal muscle, kidney and liver.

### EXAMPLE 3

Chromosome Mapping Study.

Chromosomal mapping studies were conducted using a GENEBRIDGE 4 Radiation Hybrid Panel and a Stanford G3 Radiation hybrid Panel and show that the human serine racemase gene maps to chromosome 17pl3.

Human serine racemase was mapped by polymerase chain reaction (PCR) screening of the GENEBRIDGE 4 Radiation Hybrid Panel and Stanford G3 Radiation hybrid Panel (RESEARCH GENETICS). Primers for amplification were 5′-TCA TGG TAC ATC CCA ACC AGG AG-3′ (SEQ ID NO:5) and 5′-CAA GCA TTC CTC CTC CAC CTA CA-3′ (SEQ ID NO:6) corresponding to nucleotides 446-468 and 549-571 of human serine racemase. In addition, the primers of G3PDH (5′-CCT GGC CAA GGT CAT CCA TGA CAA C-3′ (SEQ ID NO:7) and 5′-TGT CAT ACC AGG AAA TGA GCT TGA C-3′ (SEQ ID NO:8)) serve as positive control for the PCR reaction. PCR results were analyzed at http://carbon.wi.mit.edu:800/cgi-bin/rhmapper _noupload.pl and http://www -shgc.stanford.edu/RH/rhserverformew.html/.

### EXAMPLE 4

Assay of Serine Racemase.

Serine racemase activity is assayed as described previously (Wolosker et al., 1999b). The expressed serine racemase is extracted from the transfected cells according to the following procedure. The transfected cells are harvested by centrifugation for 5 min at 500×g, and resuspended in the lysis buffer including 50 mM Tris-HCl (pH 8.5), 10 mM 2-mercaptoethanol, 1 mM PMSF, 1% Nonidet P-40 at 4° C. Then the cells are disrupted on ice by brief sonication. The homogenate is centrifuged at 10,000×g for 10 min. The supernatant is transferred into a new tube and measured for protein concentration by using Pierce Coomassie reagent (PIERCE CHEMICAL CO., Rockford, Ill.).

The cell extracts are incubated in Tris (50 mM, pH 8.0) buffer containing 1 mM EDTA, 2 mM DTT, 15 uM PLP and 20 mM L-serine for 0.5-8 hr at 37° C. The reaction is terminated by the addition of trichloroacetic acid (TCA; 5% final concentration), and followed by centrifugation. TCA is extracted from the supernatant with 1 ml water-saturated diethyl ether twice. The amount of D-serine produced was determined by incubation of the supernatant with D-amino acid oxidase, which generates an α-keto acid, NH₃, and hydrogen peroxide. The generation of hydrogen peroxide is quantitated by the use of peroxidase and luminol, which emits light. The luminescence is counted by a luminometer.

The enzyme activity is calculated as counts from each tube minus the counts from the boiled extract tube. The Kₘ (Michaelis constant), Vₘₐₓ (Velocity), and other kinetic constants are determined for human serine racemase using standard methods commonly applied in the art.

### EXAMPLE 5

Screening for Compounds that Alter the Activity of Serine Racemase

A screening strategy is developed to specifically discover a compound from a chemical compound collection. The assays of the present invention can be adapted for high throughput screening in microtiter plate, microwell and droplet formats.

In the simplest assay, samples containing serine racemase activity are prepared and incubated with a chemical compound prior to and/or during the determination of serine racemase activity. The samples, e.g., cells, disrupted cells or cell extracts, can be prepared from cells expressing recombinant serine racemase including transformed cells, transfected cells or cells derived from transgenic animals. The concentration of the compound used can be varied across a number of samples. If a preincubation is preferred, that step can be performed for various times and often 5-10 minutes is appropriate. The samples are then assayed for serine racemase activity. One can, if desired, use the procedure described in Example 4 to determine the activity of the serine racemase enzyme.

The basal level of serine racemase activity can be determined in samples prepared from appropriate cells including cells that have not been transformed or transfected. The percent inhibition of the serine racemase activity can be determined in samples prepared from cells expressing recombinant serine racemase in presence of a compound and compared with the maximum activity determined in sample in the absence of a compound. Typically, the IC50, the concentration of a compound required to reduce the enzyme activity in a sample by half, is used to compare the potency of the compounds.

Control assays can be performed on samples prepared from recombinant cells and, if desired, non-recombinant cells. In one control assay, a cell line known to have no serine racemase activity can by contacted with the compound. Alternatively, an assay can be performed on a sample from recombinant cells expressing serine racemases activity where no compound is contacted with the sample. It may also be preferred to use samples from a cell line that does not express serine racemase and samples from the same cell line transformed or transfected to express recombinant serine racemase. These and other controls will be apparent to those of skill in the art.

### EXAMPLE 6

Assays Measuring NMDA Receptor Activity

D-serine produced by serine racemase is a co-activator of the NMDA receptors acting at the glycine site. Therefore, one can assay for compounds that affect serine racemase activity by measuring the activation of NMDA receptors. One of skill in the art will appreciate that a wide variety of assays used to measure an intracellular second messager, such as calcium, are applicable to measuring activation of NMDA receptors. Of particular interest is the use of aequorin, green fluorescent protein, or calcium sensitive dyes to generate a fluorescent signal upon activation of a NMDA receptor that produces a calcium influx.

In an assay that measures NMDA receptor activation as an indication of serine racemase activity, it can be useful to create a cell line that is recombinant for both the NMDA receptor and the serine racemase. If an aequorin based signal generation system is to be used, the starting cell line can be one that is stably transformed with an expression construct to produce aequorin.

### EXAMPLE 7

Transgenic Animals

Transgenic animals expressing serine racemase as a transgene are provided as follows. A polynucleotide having an serine racemase nucleotide sequence, e.g., the nucleotide sequence of a cDNA or genomic DNA encoding a full length serine racemase, or a polynucleotide encoding a partial sequence of the racemase, sequences flanking the coding sequence, or both, can be combined into a vector for the integration of the polynucleotide into the genome of an animal. The serine racemase sequence can be from a human serine racemase or from the animal's serine racemase.

In this example, the target cell for transgene introduction is a murine embryonic stem cell (ES). ES cells can be obtained from pre-implantation embryos of a variety of non-human animals cultured in vitro and fused with embryos (M. J. Evans et al., Nature 292:154-156 (1981); Bradley et al., Nature 309:255-258 (1984); Gossler et al. Proc. Natl. Acad. Sci. USA 83:9065-9069 (1986); and Robertson et al., Nature 322:445-448 (1986)).

The transgene is introduced into the murine ES cells by microinjection, however, a variety of standard techniques such as DNA transfection, or retrovirus-mediated transduction can be used. The injected ES cells are then combined with blastocysts from a non-human animal. The introduced ES cells colonize the embryo and contribute to the germ line of the resulting chimeric animal (R. Jaenisch, Science 240: 1468-1474 (1988)). The chimeric mice are screened for individuals in which germline transformation has occurred. These are crossed to produce animals homozygous for the transgene.

The targeted recombination events as well as the resulting mice are evaluated by techniques well known in the art, including but not limited to DNA (Southern) hybridization to detect the targeted allele, polymerase chain reaction (PCR), polyacrylamide gel electrophoresis (PAGE) and Western blots to detect DNA, RNA and protein.

Three basic types of transgenic animals are created depending on the construction of the transgene vector. If the vector is designed to include a nucleotide sequence that encodes a full length human serine racemase and to integrate at a site other than the animal's endogenous serine racemase gene, the resultant transgenic animal will express both a native and human serine racemases. If the vector is designed without a cognate serine racemase gene and to integrate at the site of the animal's endogenous serine racemase gene such that after integration the endogenous gene is altered to such an extent that the animal lacks a functional serine racemase, then a knockout animal is produced. Finally, if the vector is designed to replace the endogenous serine racemase gene with a human gene, or is designed to change the sequence of the endogenous gene to encode the amino acid sequence of the human gene, i.e., is humanized, then the resultant animal lacks a native serine racemase and expresses a human serine racemase. Animals having a human gene and lacking an endogenous gene can also be created by crossing the first type of animal with a knockout animal to obtain animals homozygous for the knockout and homozygous for the added human serine racemase gene. This can be facilitated if the human gene integrates in a chromosome different from the chromosome carrying the endogenous serine racemase gene.

Transgenic animals are a source of cells and tissues for use in assays of serine racemase modulation, activation or inhibition. Cells can be removed from the animals, established as cell lines and maintained in culture as convenient.

### REFERENCES

Altamura, C., et al. 1995. Plasma concentrations of excitatory amino acids, serine, glycine, taurine and histidine in major depression. Eur Neuropsychopharmacol. 5:71-75.

Atschul, S. F. et al. 1990. J Molec Biol 215:403.

Bishop, M. J. (ed.), 1994. Guide to Huge Computers, Academic Press, San Diego.

Bradley et al. 1984. Nature 309:255-258.

Carillo, H., and Lipton, D. 1988. SIAM J Applied Math 48:1073.

Carlton, S. M., and Zhou, S. 1998. Attenuation of formalin-induced nociceptive behaviors following local peripheral injection of gabapentin. Pain 76:201-207.

Coderre, T. J. 1993. Potent analgesia induced in rats by combined action at PCP and polyamine recognition sites of the NMDA receptor complex. Eur-J-Neurosci. 5:390-393.

Dalkara E. G., et al. 1990. Glycine, alanine and serine potentiate glutamate neurotoxicity in cerebral ischemia via NMDA receptor. Eur J Pharmacol (suppl) 183:476.

Daugherty, et al. 1991. Nucleic Acids Res. 19:2471

Danysz, W., and Parsons, C. G. 1998. Glycine and N-methyl-D-Aspartate receptors: physiological significance and possible therapeutic applications. Pharmac. Rev. 50:597-664.

Devereux, J. et al. 1984. Nucleic Acids Research 12(1):387.

Dickenson, A. H. 1990. A cure for wind up: NMDA receptor antagonists as potential analgesics. Trends Pharmacol. Sci. 11:307-309.

Evans, M. J., et al. 1981. Nature 292:154-156.

Gillman et al. 1979. Gene 8:81.

Gossler et al. 1986. Proc. Natl. Acad. Sci. USA 83:9065-9069.

Gribskov, M. and Devereux, J., (eds.). 1991. SEQUENCE ANALYSIS PRIMER, M Stockton Press, New York.

Griffin, A. M., and Griffin, H. G., (eds.). 1994. COMPUTER ANALYSIS OF SEQUENCE DATA, PART I, Humana Press, New Jersey.

Hashimoto, A. and Oka, T. 1997. Free D-aspartate and D-serine in the mammalian brain and periphery. Prog Neurobiol. 52:325-353.

Hirai, H., and Okada, Y. 1993. Serine released from hippocampal slices during deprivation of oxygen and glucose enhances the effects of glutamate on neuronal function. Neuroscience 54:61-67.

Innis (ed.). 1990. PCR Protocols: A Guide to Methods and Applications, Academic Press, New York, N.Y.

Ivanovic, A., et al. 1998. Expression and initial characterization of a soluble glycine binding domain of the N-methyl-D-aspartate receptor NR1 subunit. J Biol. Chem. 273:19933-19937.

Jaenisch, R. 1988. Science 240:1468-1474.

Javitt, D. C. and Zukin, S. R. 1991. Recent advances in the phencyclidine model of schizophrenia. Am J Psychiatry 148:1301-1308.

Jun, J. H., and Yaksh, T. L 1998. The effect of intrathecal gabapentin and 3-isobutyl gamma-aminobutyric acid on the hyperalgesia observed after thermal injury in the rat. Anesth. Analg. 86:348-354.

Kanthan, R., et al. 1995. Intracerebral human microdialysis: in vivo study of an acute focal ischemic model of the human brain. Stroke 26:870-873.

Kolhekar, R., and Gebhart, G. F. 1996. Modulation of spinal visceral nociceptive transmission by NMDA receptor activation in the rat. J Neurophysiol. 75:2344-2353.

Lesk, A. M., (ed.). 1988. COMPUTATIONAL MOLECULAR BIOLOGY, Oxford University Press, New York.

Miyazaki, J., et al. 1999. Expression and characterization of a glycine-binding fragment of the N-methyl-D-aspartate receptor subunit NR1. Biochem. J. 340:687-692.

Needleham, et al. 1970. J. Mol. Biol. 48:443-453.

Roberts et al. 1987. Nature 328:731.

Robertson et al. 1986. Nature 322:445-448.

Ronneengstrom, E., et al. 1992. Intracerebral microdialysis of extracellular amino acids in the human epileptic focus. J Cereb Blood Flow Metab. 12:873-876.

Saigoh, K., et al. 1998. The stereo-specific effect of D-serine ethylester and the D-cycloserine in ataxic mutant mice. Brain Research 808:42-47.

Saiki, et al. 1988. Science 239:487.

Sambrook, et al. 1989, Molecular Cloning: A Laboratory Manual, second edition, Cold Spring Harbor Laboratory Press, Cold Spring Harbor, N.Y.

Sankoff, et al. 1983. in Time Warps, String Edits, and Macromolecules: The Theory and Practice of Sequence Comparison, Addison-Wesley, Reading, Mass.

Schell, M. J., et al. 1997a. D-serine as a neuromodulator: regional and developmental localizations in rat brain glia resemble NMDA receptors. J Neurosci. 17:1604-1615.

Schell, M. J., et al. 1997b. D-serine, an endogenous synaptic modulator: localization to astrocytes and glutamate-stimulated release. Proc Natl Acad Sci USA 92:3948-3952.

Smith, D. W. (ed.), 1993. BIOCOMPUTING: INFORMATICS AND GENOME PROJECTS, Academic Press, New York.

Tsai, G., et al. 1998. D-serine added to antipsychotics for the treatment of schizophrenia. Biol. Psych. 44:1081-1089.

von Heinje, G. 1987. SEQUENCE ANALYSIS IN MOLECULAR BIOLOGY, von Heinje, G., Academic Press.

Wolosker, H., et al. 1999. Serine racemase: a glial enzyme synthesizing D-serine to regulate glutamate-N-methyl-D-aspartate neurotransmission. Proc Natl Acad Sci USA 96:13409-13414.

Wolosker, H., et aL 1999b. Purification of serine racemase: biosynthesis of the neuromodulator D-serine. Proc Natl Acad Sci USA 96:721-725.

The Examples have been provided as guidance in practicing the invention and are not limiting of the scope of the invention which is defined by the following claims.

What is claimed:

1. A recombinant polynucleotide selected from the group consisting of:
(a) a polynucleotide encoding a polypeptide having an amino acid sequence of SEQ ID NO:2.
(b) a polynucleotide having the nucleotide sequence of SEQ ID NO:1,
(c) a polynucleotide which is complementary to the polynucleotide of (a) or (b), and
(d) a polynucleotide that hybridizes with a polynucleotide of (a), (b), or (c) under stringent conditions.

2. The polynucleotide of claim 1 wherein the polynucleotide comprises nucleotides selected from the group consisting of natural, non-natural and modified nucleotides.

3. The polynucleotide of claim 1 wherein the internucleotide linkages are selected from the group consisting of natural and non-natural linkages.

4. An expression vector that directs the expression of a polynucleotide selected from the group consisting of:
(a) a polynucleotide encoding a polypeptide having an amino acid sequence of SEQ ID NO:2.
(b) a polynucleotide having the nucleotide sequence of SEQ ID NO: 1,
(c) a polynucleotide which is complementary to the polynucleotide of (a) or (b), and
(d) a polynucleotide that hybridizes with a polynucleotide of (a), (b), or (c) under stringent conditions.

5. A host cell comprising the expression vector of claim 4.

6. A process for expressing a serine racemase protein from a recombinant host cell, comprising:
(a) transforming a suitable host cell with an expression vector of claim 4; and,
(b) culturing the host cell of step (a) in conditions under which allow expression of said the serine racemase protein from said expression vector.

7. A recombinant polypeptide having an amino acid sequence of SEQ ID NO:2.

8. A method of determining whether a candidate compound is an inhibitor of a serine racemase polypeptide comprising:
(a) providing at least one host cell harboring an expression vector that includes a polynucleotide selected from the group consisting of:
(i) a polynucleotide encoding a polypeptide having an amino acid sequence of SEQ ID NO:2, and
(ii) a polynucleotide having the coding sequence from SEQ ID NO: 1,
(b) contacting at least one of said cells with the candidate to permit the interaction of the candidate with the serine racemase polypeptide, and
(c) determining whether the candidate is an inhibitor of the serine racemase polypeptide by ascertaining the relative activity of the polypeptide in the presence of the candidate.

9. The method of claim 8 wherein in step (c) the relative activity is determined by comparing a measurement of serine racemase polypeptide activity of at least one cell before step (b) to a measurement of serine racemase polypeptide activity of at least one cell after step (b).

10. The method of claim 8 further comprising a control assay using a serine racemase polypeptide that is not contacted with a candidate.

11. A transgenic animal lacking a functional endogenous serine racemase gene.

12. The animal of claim 12 further comprising a human serine racemase gene.

13. The animal of claim 12 wherein the activity of the human serine racemase is detectable in a homogenate of neural tissue in the absence of the activity of the endogenous serine racemase.

14. A cell line derived from an animal according to claim 13.


SEQUENCE LISTING (8 sequences)

SEQ ID NO: 1 - DNA, 1023 bp, Homo Sapien
atgtgtgctcagtattgcatctcctttgctgatgttgaaaaagctcatatcaacattcgagattctatccacctcacaccagtgctaacaagctccattttgaatcaactaacagggcgcaatcttttcttcaaatgtgaactcttccagaaaacaggatcttttaagattcgtggtgctctcaatgccgtcagaagcttggttcctgatgctttagaaaggaagccgaaagctgttgttactcacagcagtggaaaccatggccaggctctcacctatgctgccaaattggaaggaattcctgcttatattgtggtgccccagacagctccagactgtaaaaaacttgcaatacaagcctacggagcgtcaattgtatactgtgaacctagtgatgagtccagagaaaatgttgcaaaaagagttacagaagaaacagaaggcatcatggtacatcccaaccaggagcctgcagtgatagctggacaagggacaattgccctggaagtgctgaaccaggttcctttggtggatgcactggtggtacctgtaggtggaggaggaatgcttgctggaatagcaattacagttaaggctctgaaacctagtgtgaaggtatatgctgctgaaccctcaaatgcagatgactgctaccagtccaagctgaaggggaaactgatgcccaatctttatcctccagaaaccatagcagatggtgtcaaatccagcattggcttgaacacctggcctattatcagggaccttgtggatgatatcttcactgtcacagaggatgaaattaagtgtgcaacccagctggtgtgggagaggatgaaactactcattgaacctacagctggtgttggagtggctgctgtgctgtctcaacattttcaaactgtttccccagaagtaaagaacatttgtattgtgctcagtggtggaaatgtagacttaacctcctccataacttgggtgaagcaggctgaaaggccagcttcttatcagtctgtttctgtttaa

SEQ ID NO: 2 - protein, 340 aa, Homo Sapien
MCAQYCISFADVEKAHINIRDSIHLTPVLTSSILNQLTGRNLFFKCELFQKTGSFKIRGALNAVRSLVPDALERKPKAVVTHSSGNHGQALTYAAKLEGIPAYIVVPQTAPDCKKLAIQAYGASIVYCEPSDESRENVAKRVTEETEGIMVHPNQEPAVIAGQGTIALEVLNQVPLVDALVVPVGGGGMLAGIAITVKALKPSVKVYAAEPSNADDCYQSKLKGKLMPNLYPPETIADGVKSSIGLNTWPIIRDLVDDIFTVTEDEIKCATQLVWERMKLLIEPTAGVGVAAVLSQHFQTVSPEVKNICIVLSGGNVDLTSSITWVKQAERPASYQSVSV

SEQ ID NO: 3 - DNA, 23 bp, Homo Sapien
cttgcaatacaagcctacggagc

SEQ ID NO: 4 - DNA, 24 bp, Homo Sapien
gttcaagccaatgctggatttgac

SEQ ID NO: 5 - DNA, 23 bp, Homo Sapien
tcatggtacatcccaaccaggag

SEQ ID NO: 6 - DNA, 23 bp, Homo Sapien
caagcattcctcctccacctaca

SEQ ID NO: 7 - DNA, 25 bp, Homo Sapien
cctggccaaggtcatccatgacaac

SEQ ID NO: 8 - DNA, 25 bp, Homo Sapien
tgtcataccaggaaatgagcttgac